(19) United States
(12) Patent Application Publication
(10) Pub. No.: US 2018/0103853 A1
(43) Pub. Date: Apr. 19, 2018

(54) METHOD, APPARATUS, AND PROGRAM FOR OUTPUTTING INDEX

(71) Applicant: NIHON KOHDEN CORPORATION, Shinuku-ku, Tokyo (JP)

(72) Inventors: Haruka MORIMOTO, Shinjuku-ku, Tokyo (JP); Teiji UKAWA, Shinjuku-ku, Tokyo (JP); Takehito SUZUKI, Shinjuku-ku, Tokyo (JP)

(21) Appl. No.: 15/573,646

(22) PCT Filed: May 11, 2016
(86) PCT No.: PCT/JP2016/002311

§ 371 Date: Nov. 13, 2017

(30) Foreign Application Priority Data
May 14, 2015 (JP) 2015-099352

(51) Int. Cl. A61B 5/02; A61B 5/021

(52) U.S. Cl. CPC A61B 5/02007; A61B 5/02225; A61B 5/02116

(57) ABSTRACT

A method for outputting an index for determining a condition of a blood vessel includes acquiring a diastolic blood pressure for each of upper and lower limbs of a living body, calculating a ratio of the diastolic blood pressure acquired in the acquiring step of the lower limb to the diastolic blood pressure acquired in the acquiring step of the upper limb, and outputting the ratio of the diastolic blood pressures calculated in the calculating step.

### TECHNICAL FIELD

The present invention relates to a method, apparatus, and program for outputting an index.

### BACKGROUND ART

As an index for diagnosing stenosis or occlusion of a blood vessel due to arterial sclerosis, the ABI (Ankel-Brachial pressure Index) which is a ratio of the systolic blood pressures of the upper arm and the ankle joint (ankle) is used. Generally, the blood pressure of the lower limb is higher than that of the upper limb. Therefore, the value of the ABI is often larger than 1.0. In the case where the value of the ABI is larger than 0.9 and smaller than 1.4, usually, it is determined that the condition of a blood vessel is normal, and, in the case where the value of the ABI is equal to or smaller than 0.9, it is determined that there is suspicion of stenosis or occlusion of a blood vessel. JP-A-2006-314613 discloses a technique for diagnosing the condition of a blood vessel in consideration of the ABI, a ratio of pulse pressures (hereinafter, referred to as ppABI), and a ratio of mean blood pressures (hereinafter, referred to as mABI).

### SUMMARY

### Technical Problem

In the case where calcification of a blood vessel is caused to occur by progress of arterial sclerosis, dialytic treatment of diabetes, or the like, however, the systolic blood pressure, the mean blood pressure, and the pulse pressure cannot be appropriately measured. In the technique disclosed in JP-A-2006-314613, therefore, there is a problem in that, in such a case, the condition of a blood vessel cannot be adequately determined.

Firstly, the systolic blood pressure by using oscillometric method is acquired based on the maximum value of a pulse wave amplitude in the case where the mean blood pressure is acquired. Specifically, a pulse wave amplitude having a magnitude which is obtained by subtracting a predetermined rate from the maximum value of a pulse wave amplitude is calculated as the pulse wave amplitude at the systolic blood pressure. The predetermined rate can be adequately set as a value according to various conditions such as the configuration of a measuring device, and the measurement environment. After a pulse wave amplitude which varies in accordance with the change of the cuff pressure shows the maximum value, the cuff pressure at a timing when the pulse wave amplitude is lowered to the pulse wave amplitude at the calculated systolic blood pressure is acquired as the systolic blood pressure.

In the case where calcification of a blood vessel occurs, the flexibility and elasticity of the blood vessel are impaired, and, even when the blood vessel is compressed by pressurization of the cuff, there is hence a case where the blood vessel is not completely closed. In this case, even when the cuff pressure is raised, a state where the pulse wave amplitude is high is maintained, and the cuff pressure at which the pulse wave amplitude at the systolic blood pressure is detected is increased. As a result, the systolic blood pressure is measured as a value which is larger than the true value. In the case where calcification progresses in a blood vessel of the lower limb, therefore, the systolic blood pressure of the lower limb is measured as a value which is larger than the true value, and also the value of the ABI is calculated as a value which is larger than the true value. Consequently, the condition of the blood vessel cannot be adequately determined by using the ABI, and this is problematic. The pulse pressure is the deference between the systolic blood pressure and the diastolic blood pressure. In the case where the systolic blood pressure cannot be adequately measured as described above, therefore, also the pulse pressure cannot be adequately acquired, and similarly this is problematic.

Similarly with the measurement of the systolic blood pressure, for example, the cuff is pressurized and then depressurized, and the mean blood pressure is measured as the cuff pressure at a timing when the pulse wave amplitude is maximum. In the case where calcification of a blood vessel occurs, the flexibility and elasticity of the blood vessel are impaired, and, even when the cuff pressure is lowered, there is hence a case where the pulse wave amplitude does not form a distinct peak, and remains in the vicinity of the maximum value. In this case, it is difficult to determine the timing at which the cuff pressure is acquired as the mean blood pressure, and there is a possibility that the value of the mean blood pressure is not adequately acquired. Consequently, the condition of the blood vessel cannot be adequately determined by using the mABI, and this is problematic.

The invention has been conducted in view of the above-discussed circumstances. It is an object of the invention to provide a method, apparatus, and program for outputting an index for adequately determining the condition of a blood vessel even in the case where calcification of the blood vessel occurs.

The object is achieved by the following means.

The method for outputting an index for determining a condition of a blood vessel has an acquiring step, a calculating step, and an outputting step. In the acquiring step, a diastolic blood pressure is acquired for each of upper and lower limbs of the living body. In the calculating step, a ratio of the diastolic blood pressure of the lower limb to the diastolic blood pressure of the upper limb is calculated, the diastolic blood pressure of the lower limb and the diastolic blood pressure of the upper limb being acquired in the acquiring step. In the outputting step, the ratio of the diastolic blood pressures which is calculated in the calculating step is output.

The apparatus for outputting an index for determining a condition of a blood vessel has an acquiring section, a calculating section, and an outputting section. The acquiring section acquires a diastolic blood pressure for each of upper and lower limbs of the living body. The calculating section calculates a ratio of the diastolic blood pressure of the lower limb to the diastolic blood pressure of the upper limb, the diastolic blood pressure of the lower limb and the diastolic blood pressure of the upper limb being acquired by the acquiring section. The outputting section outputs the ratio of the diastolic blood pressures which is calculated by the calculating section.

The program for outputting an index for determining a condition of a blood vessel causes a computer to execute an acquiring step, a calculating step, and an outputting step. In the acquiring step, a diastolic blood pressure is acquired for each of upper and lower limbs of the living body. In the calculating step, a ratio of the diastolic blood pressure of the lower limb to the diastolic blood pressure of the upper limb is calculated, the diastolic blood pressure of the lower limb and the diastolic blood pressure of the upper limb being acquired in the acquiring step. In the outputting step, the ratio of the diastolic blood pressures which is calculated in the calculating step is output.

### Advantageous Effects of Invention

In accordance with an embodiment of this invention, the apparatus for outputting an index calculates and outputs a ratio of the diastolic blood pressure of the lower limb to the diastolic blood pressure of the upper limb, as an index for determining the condition of a blood vessel. The ratio of diastolic blood pressures is hardly affected by calcification of a blood vessel. Even when calcification occurs, therefore, it is possible to output an index by which the condition of a blood vessel can be adequately determined. While combining the systolic blood pressure which is easily affected by calcification, and the diastolic blood pressure which is hardly affected by calcification, evaluation is performed. Therefore, it is possible to determine more correctly the degree of calcification. Moreover, stenosis/occlusion and calcification can be simultaneously determined by one measurement.

### BRIEF DESCRIPTION OF DRAWINGS

[FIG. 1]

FIG. 1 is a diagram schematically illustrating the configuration of an index outputting apparatus of a first embodiment of the invention.

[FIG. 2A]

FIG. 2A illustrates a method for measuring blood pressures.

[FIG. 2B]

FIG. 2B illustrates a method for measuring blood pressures.

[FIG. 2C]

FIG. 2C illustrates a method for measuring blood pressures.

[FIG. 3]

FIG. 3 illustrates vascular pressure-volume curves of a normal blood vessel and a calcified blood vessel.

[FIG. 4]

FIG. 4 is a flowchart illustrating the procedures of processes in the index outputting apparatus.

[FIG. 5]

FIG. 5 illustrates an example of a screen which is displayed on the index outputting apparatus of the first embodiment.

[FIG. 6]

FIG. 6 illustrates an example of a screen which displays combinations of a ratio of diastolic blood pressures, and that of systolic blood pressures.

[FIG. 7]

FIG. 7 illustrates an example of a screen which displays combinations of a ratio of diastolic blood pressures, and that of mean blood pressures.

[FIG. 8]

FIG. 8 illustrates an example of a screen which displays combinations of a ratio of diastolic blood pressures, and that of pulse pressures.

[FIG. 9]

FIG. 9 illustrates an example of a method for determining the condition of a blood vessel based on a plat region in the screen illustrated in FIG. 6.

[FIG. 10]

FIG. 10 illustrates an example of a method for determining the condition of a blood vessel based on a plot region.

### DESCRIPTION OF EMBODIMENTS

Hereinafter, embodiments of the invention will be described with reference to the accompanying drawings. In the description of the drawings, the identical components are denoted by the same reference numerals, and duplicated description is omitted. In the drawings, the dimension ratios are exaggerated for the sake of convenience in description, and may be sometimes different from the actual ratios.

### Example 1

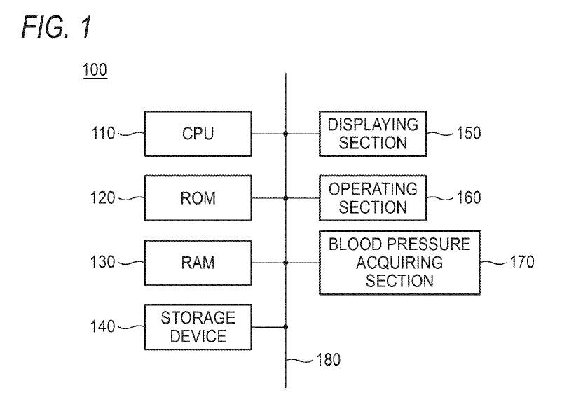

FIG. 1 is a diagram schematically illustrating the configuration of an index outputting apparatus of a first embodiment of the invention.

As illustrated in FIG. 1, the index outputting apparatus 100 may include a CPU 110, a ROM 120, a RAM 130, a storage device 140, a displaying section 150, an operating section 160, and a blood pressure acquiring section 170. These components are connected to one another through a bus 180 for exchanging signals.

The CPU 110 controls the above-mentioned components and performs various calculation processes in accordance with programs which are recorded in the ROM 120 and the storage device 140. The CPU 110 executes the programs to function as a calculating section and a determining section. The ROM 120 stores various programs and data. The RAM 130 functions as a working area to temporarily store programs and data.

The storage device 140 stores various programs including an operating system, and various data. The storage device 140 further stores various thresholds for determining the condition of a blood vessel based on indexes.

The displaying section 150 is, for example, a liquid crystal display, and displays various kinds of information such as the name of a subject, measure blood pressures, and calculated indexes.

The operating section 160 is used for performing various inputs. The operating section 160 may include operation keys which are realized as software by a touch panel on the displaying section 150, operation buttons which are disposed as hardware, and the like.

The blood pressure acquiring section 170 is connected to cuffs, probes, or the like which are used for measuring the blood pressure, and acquires blood pressure values such as the diastolic blood pressure of the subject. For example, the blood pressure acquiring section 170 acquires the diastolic blood pressure of the upper limb from a cuff which is attached to the upper arm of the subject, and that of the lower limb from a cuff which is attached to the ankle joint of the subject. The blood pressure acquiring section 170 may acquire the blood pressures of the right and left upper arms and ankle joints of the subject, or acquire those of the right or left upper arm and ankle joint. Alternatively, the blood pressure acquiring section 170 may sequentially acquire the blood pressures of measurement portions by using a single cuff, or acquire at one time the blood pressures of a plurality of measurement portions by using a plurality of cuffs. The method for acquiring blood pressures will be described in detail later.

The index outputting apparatus 100 may include components other than the above-described components, or may not include a part of the above-described components.

The thus configured index outputting apparatus 100 acquires the diastolic blood pressure of the upper limb of the living body, and that of the lower limb, and calculates and outputs a ratio of the diastolic blood pressure of the lower limb to the diastolic blood pressure of the upper limb (hereinafter, the ratio is referred to as the dABI), as an index for determining the condition of a blood vessel. Hereinafter, the function of the index outputting apparatus 100 will be described.

Firstly, a method for measuring blood pressures including the diastolic blood pressure, and advantages of the use of the dABI as an index for determining the condition of a blood vessel will be described.

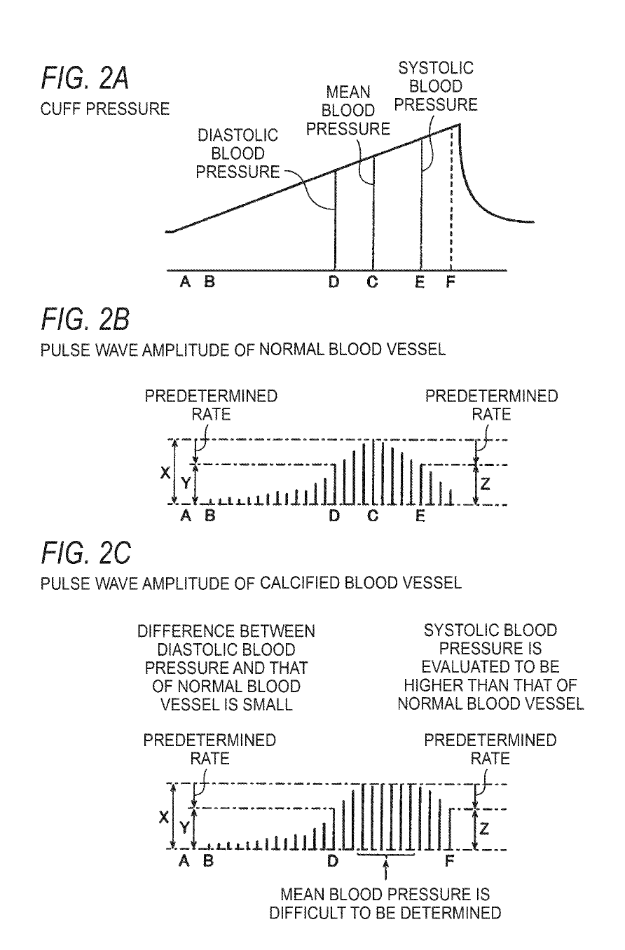

FIGS. 2A to 2C are views illustrating a method for measuring blood pressures.

FIGS. 2A to 2C illustrate relationships between the pressure of a cuff attached to a measurement portion of the subject, such as the upper arm, and the pulse wave amplitude detected by the cuff, with respect to a normal blood pressure and a calcified blood vessel. FIG. 2A illustrates the magnitude of the cuff pressure, FIG. 2B illustrates the pulse wave amplitude which is detected in a normal blood vessel in response to the cuff pressure illustrated in FIG. 2A, and FIG. 2C illustrates the pulse wave amplitude which is detected in a calcified blood vessel in response to the cuff pressure illustrated in FIG. 2A.

In the embodiment, as illustrated in a range from the left side to the right side of the graph of FIG. 2A, the cuff pressure is gradually raised from a state where the pressure is zero, and the blood pressure is measured based on the pulse wave amplitude which is detected by the cuff during the pressurization. In the embodiment, the mean blood pressure is firstly measured, and then the diastolic blood pressure and the systolic blood pressure are measured by using the pulse wave amplitude at the timing when the mean blood pressure is measured.

Referring to FIG. 2B, firstly, the measurement of the mean blood pressure, diastolic, blood pressure, systolic blood pressure, and pulse pressure in a normal blood vessel will be described.

The mean blood pressure is measured by acquiring the cuff pressure at the timing when the pulse wave amplitude is maximum. As indicated at the timing A of the graph of FIG. 2B, when the cuff pressure is low, the cuff is not sufficiently contacted with the upper arm or the like, and therefore the pulse wave is not detected. As indicated at the timing B of FIG. 2B, when the cuff pressure reaches a certain magnitude, the pulse wave begins to be detected. For a short period thereafter, in accordance with the rise of the cuff pressure, also the detected pulse wave amplitude is increased. As indicated at the timing C of FIG. 2B, when the cuff pressure is sufficiently raised, and the internal and external pressures of the blood vessel are equal to each other, the pulse wave amplitude reaches the maximum value. The cuff pressure at the timing C is acquired as the mean blood pressure. The determination of whether the pulse wave amplitude reaches the maximum value or not is performed by, for example, finding a situation where the maximum value of the pulse wave amplitude is not updated for a constant period of time. In the determination, the increasing/decreasing amount, rate, pattern, and the like of the pulse wave amplitude may be considered. Thereafter, the cuff pressure is further raised, the blood vessel is compressed, and the pulse wave amplitude is lowered.

The diastolic blood pressure is acquired based on the maximum value of the pulse wave amplitude at the timing when the mean blood pressure is acquired. Specifically, firstly, a pulse wave amplitude having a magnitude which is obtained by subtracting a predetermined rate from the maximum value of the pulse wave amplitude is calculated as the pulse wave amplitude at the diastolic blood pressure. The predetermined rate can be adequately set as a value according to various conditions such as the configuration of a measuring device, and the measurement environment. The cuff pressure at a timing when the pulse wave amplitude which varies in accordance with the change of the cuff pressure firstly reaches the pulse wave amplitude at the calculated diastolic blood pressure is acquired as the diastolic blood pressure. In the example of FIG. 2B, the maximum value of the pulse wave amplitude at the timing when the mean blood pressure is acquired is X, and the magnitude which is obtained by subtracting the predetermined rate of X from X is Y. Therefore, the cuff pressure at the timing D when the pulse wave amplitude reaches Y before the amplitude reaches the maximum value is acquired as the diastolic blood pressure. In the embodiment, as described above, the diastolic blood pressure can be acquired at the timing when the mean blood pressure is acquired.

Similarly with the diastolic blood pressure, the systolic blood pressure is acquired based on the maximum value of the pulse wave amplitude at the timing when the mean blood pressure is acquired. Specifically, a pulse wave amplitude having a magnitude which is obtained by subtracting a predetermined rate from the maximum value of the pulse wave amplitude is calculated as the pulse wave amplitude at the systolic blood pressure. The predetermined rate can be adequately set as a value according to various conditions such as the configuration of a measuring device, and the measurement environment. The cuff pressure at a timing when the pulse wave amplitude which varies in accordance with the change of the cuff pressure is lowered to the pulse wave amplitude at the calculated systolic blood pressure after the amplitude reaches the maximum value is acquired as the systolic blood pressure. In the example of FIG. 2B, the maximum value of the pulse wave amplitude at the timing when the mean blood pressure is acquired is X, and the magnitude which is obtained by subtracting the predetermined rate of the mean blood pressure from X is Z. Therefore, the cuff pressure at the timing E when the pulse wave amplitude reaches Z after the amplitude reaches the maximum value is acquired as the systolic blood pressure. In the embodiment, as described above, the systolic blood pressure can be acquired at the timing when, after that when the mean blood pressure is acquired, the cuff pressure is further raised, and the pulse wave amplitude is Z.

The pulse pressure is acquired as the difference between the thus obtained systolic and diastolic blood pressures.

Next, the case where blood pressures of a calcified blood vessel are measured will be described while comparing with the case where blood pressures are measured in a normal blood vessel.

Firstly, the mean blood pressure will be described. In a normal blood vessel, as indicated at the timing C of FIG. 2B, a distinct peak of the increase and decrease of the pulse wave amplitude is formed. This allows the timing when the pulse wave amplitude is maximum, to be easily specified, and therefore the mean blood pressure can be clearly determined. This is because, in a normal blood vessel, the flexibility and elasticity of the blood vessel are maintained, and the response of the blood vessel to a change of the cuff pressure is good. In a calcified blood vessel, as illustrated in of FIG. 2C, by contrast, the pulse wave amplitude remains high in the vicinity of the maximum value, and a distinct peak is not formed. This is because the flexibility and elasticity of the blood vessel are impaired, and therefore the response of the blood vessel to a change of the cuff pressure is not sensitive. In a calcified blood vessel, therefore, the timing when the pulse wave amplitude reaches the maximum value cannot be clearly determined, and it is difficult to correctly determine the mean blood pressure.

Next, the systolic blood pressure will be described. In a normal blood vessel, when the cuff pressure is raised in order to measure the systolic blood pressure, as indicated in the range from the timing C to the timing E of FIG. 2B, the artery is adequately compressed, and the pulse wave amplitude is smoothly lowered. In a calcified blood vessel, when the cuff pressure is raised in order to measure the systolic blood pressure, as illustrated in the right side of FIG. 2C, by contrast, the artery is not adequately compressed, and therefore the lowering degree of the pulse wave amplitude is small as compared with that in the case of a normal blood vessel. In a calcified blood vessel, even when the cuff pressure is raised, namely, the state where the pulse wave amplitude is high is maintained. In a calcified blood vessel, as a result, the timing when the pulse wave amplitude has a magnitude which is obtained by subtracting the predetermined rate from the maximum value is shifted to the timing F which is higher side of the cuff pressure, as compared with the case of a normal blood vessel. In a calcified blood vessel, therefore, the systolic blood pressure is measured as a value which is larger than the true value.

With respect to the diastolic blood pressure, as illustrated in FIGS. 2B and 2C, by contrast, the difference between the value acquired from a normal blood vessel, and that acquired from a calcified blood vessel is small, and a stable result can be obtained without being affected by calcification. The reason of this will be described in detail.

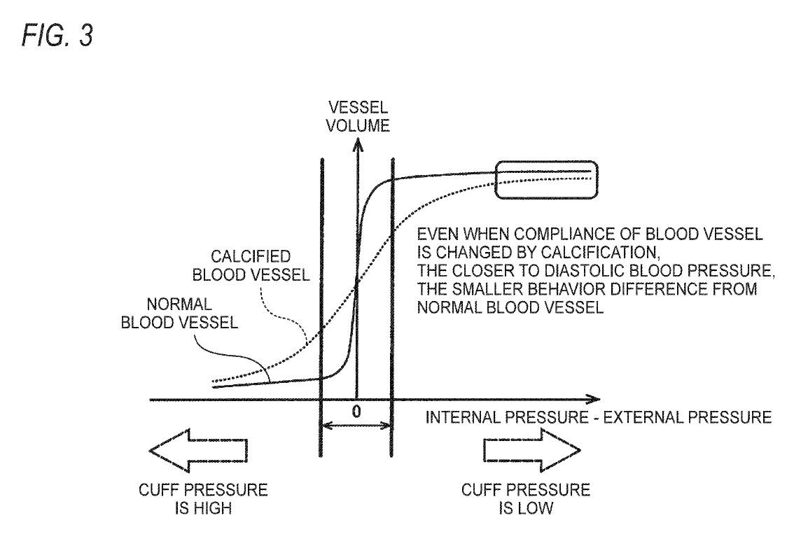

FIG. 3 illustrates vascular pressure-volume curves of a normal blood vessel and a calcified blood vessel.

The abscissa in FIG. 3 indicates the difference (the internal pressure—the external pressure) between the internal and external pressures of a blood vessel, and the ordinate indicates the vessel volume. The curve of the solid line shows a volume change in a normal blood vessel, and that of the broken line shows a volume change in a calcified blood vessel.

In FIG. 3, in the minus portion of the abscissa which is on the left side of zero, the value of (the internal pressure—the external pressure) is negative, and therefore the portion shows a state where the external pressure is higher than the internal pressure. This state corresponds to that where the cuff pressure is higher. In the plus portion of the abscissa which is on the right side of zero, by contrast, the value of (the internal pressure—the external pressure) is positive, and therefore the portion shows a state where the internal pressure is higher than the external pressure. This state corresponds to that where the cuff pressure is lower. In the abscissa, the further advancing toward the left side, the higher the external pressure, i.e., a state where the cuff pressure is high and close to the systolic blood pressure is shown, and, the further advancing toward the right side, the lower the external pressure, i.e., a state where the cuff pressure is low and close to the diastolic blood pressure is shown. In the embodiment, in the case where the cuff pressure is gradually raised from zero, for example, the cuff pressure transits from the right side of the graph of FIG. 3 toward the left side.

The portion of the abscissa in which the value is zero shows a state where the internal and external pressures of a blood vessel are equal to each other, and the blood pressure which is measured in this state is the mean blood pressure. The difference between the internal and external pressures of a blood vessel is continually changed by pulsation. In the state where the value of the abscissa is zero, therefore, the difference between the actual internal and external pressures is continually changed within a range indicated by the thick lines and the double-headed arrow, and the average is zero. In the case where the internal pressure of a blood vessel is caused to be high by pulsation, for example, the difference between the internal and external pressures has a value indicated by the right thick line, and also the vessel volume has correspondingly a large value. In the case where the internal pressure of a blood vessel is caused to be low by pulsation, by contrast, the difference between the internal and external pressures has a value indicated by the left thick line, and also the vessel volume has correspondingly a small value.

As shown by the curve of the solid line in FIG. 3, in the case of a normal blood vessel, in the left side of the graph in which the cuff pressure is close to the systolic blood pressure, the inclination of the curve is small, and the change rate of the vessel volume is small. In the vicinity of the middle portion of the graph in which the cuff pressure is close to the mean blood pressure, the inclination is sharply increased, and the change rate of the vessel volume is sharply increased. In the right side of the graph in which the cuff pressure is close to the diastolic blood pressure, the inclination of the curve is small, and the change rate of the vessel volume is small.

As shown by the curve of the broken line in FIG. 3, in the case of a calcified blood vessel, in the range from the left side of the graph to the vicinity of the middle of the graph, by contrast, the inclination of the curve steadily transits while maintaining a certain degree, and is largely different from that in the case of a normal blood vessel. Namely, it is shown that, in the states where the cuff pressure is close to the diastolic blood pressure and the mean blood pressure, the change rate of the vessel volume in a calcified blood vessel is largely different from that in a normal blood vessel. This is caused because, in a calcified blood vessel, the flexibility and elasticity of the blood vessel are impaired unlike in a normal blood vessel, and the blood vessel is hardened. In the case where the cuff pressure is high, therefore, the blood vessel cannot flexibly respond to the cuff pressure.

In the right side of the graph in which the cuff pressure is close to the diastolic blood pressure, also in the case of a calcified blood vessel, the inclination of the curve is small and approximately equal to the inclination in the case of a normal blood vessel. Namely, it is shown that, in the state where the cuff pressure is close to the systolic blood pressure, in a calcified blood vessel and a normal blood vessel, the difference in the change rate of the vessel volume is small, and that in blood vessel behavior is small. Also in a calcified blood vessel, in the state where the cuff pressure is close to the diastolic blood pressure, therefore, the blood pressure is hardly affected by calcification, and the blood vessel exhibits behavior similar to that of a normal blood vessel. When the dABI is used as an index for determining the condition of a blood vessel, also in a calcified blood vessel, consequently, the condition of a blood vessel can be adequately determined in a similar manner as in a normal blood vessel.

### (Summary of Processes in Index Outputting Apparatus 100)

Next, the procedures of processes in the index outputting apparatus 100 of the first embodiment will be described.

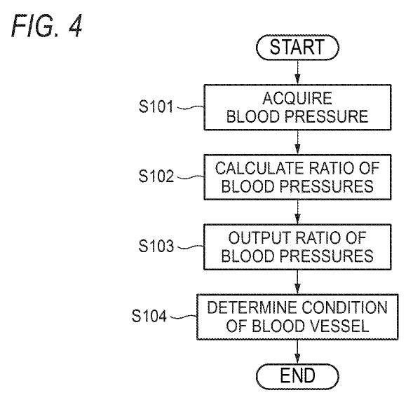

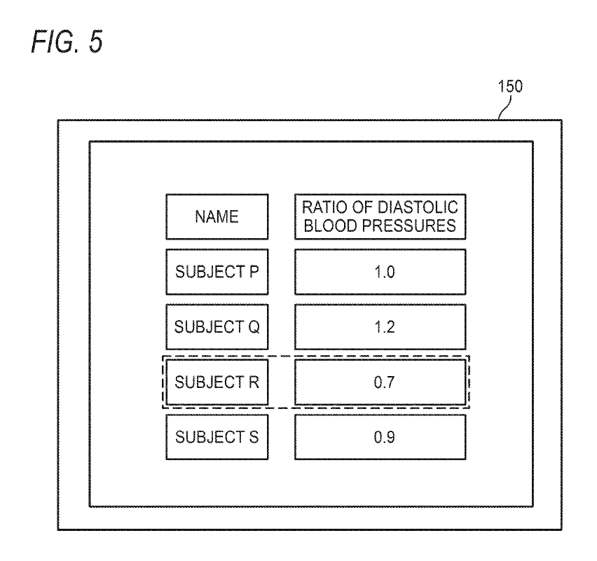

FIG. 4 is a flowchart illustrating the procedures of the processes in the index outputting apparatus, and FIG. 5 illustrates an example of a screen which is displayed on the index outputting apparatus of the first embodiment. The processes of the index outputting apparatus 100 illustrated in the flowchart of FIG. 4 are stored as programs in the storage device 140 of the index outputting apparatus 100, and executed when the CPU 110 controls the sections.

Firstly, the index outputting apparatus 100 controls the blood pressure acquiring section 170 to acquire the diastolic blood pressures of the upper and lower limbs of the subject (step S101). Specifically, the index outputting apparatus 100 acquires the diastolic blood pressures of the upper and lower limbs of the subject from measuring devices such as cuffs attached to the upper arm, ankle joint, and the like of the subject. The index outputting apparatus 100 may acquire the diastolic blood pressures of the right and left upper and lower limbs, or acquire those of the right or left upper and lower limbs. The index outputting apparatus 100 stores the acquired diastolic blood pressures of the upper and lower limbs in the storage device 140.

Then, the index outputting apparatus 100 calculates the dABI (step S102). Specifically, the index outputting apparatus 100 calculates the dABI by using the diastolic blood pressures of the upper and lower limbs which are acquired in the process of step S101. The index outputting apparatus 100 stores the calculated dABI in the storage device 140.

Then, the index outputting apparatus 100 outputs the dABI (step S103). Specifically, the index outputting apparatus 100 displays the dABI which is stored in the storage device 140 in the process of step S102, on a screen such as illustrated in FIG. 5 of the displaying section 150. In the screen example of FIG. 5, the dABI is displayed for four subjects P, Q, R, and S. For example, the user such as a medical staff can determine the condition of a blood vessel of the subject by checking the values of the dABI displayed on the screen, and comparing them with a predetermined threshold. The determination method and the threshold which is used in the determination will be described in detail later.

Then, the index outputting apparatus 100 determines the condition of a blood vessel based on the dABI (step S104). Specifically, the index outputting apparatus 100 compares the dABI which is stored in the storage device 140 in step S102, with the predetermined threshold which is previously stored in the storage device 140, thereby determining the condition of a blood vessel. The index outputting apparatus 100 displays the result of the determination on the displaying section 150. As the predetermined threshold used in the determination of the condition of a blood vessel, similarly with the known determination method using the dABI, the two values of 0.9 and 1.4 may be used as first and second thresholds. In this case, when the dABI is equal to or smaller than 0.9, it is determined that there is a possibility of stenosis or occlusion. When the dABI is larger than 0.9 and smaller than 1.4, it is determined that the condition is normal. When the dABI is equal to or larger than 1.4, it is determined that there is a possibility of an abnormality such as calcification. Alternatively, considering the tendency that the dABI has a value smaller than the ABI, values which are slightly smaller than 0.9 and 1.4, respectively, such as 0.8 and 1.3 may be set. In the alternative, when the dABI is equal to or smaller than 0.8, it is determined that there is a possibility of stenosis or occlusion. When the dABI is larger than 0.8 and smaller than 1.3, it is determined that the condition is normal. When the dABI is equal to or larger than 1.3, it is determined that there is a possibility of an abnormality such as calcification. The values and numbers of the thresholds are not limited to those of the above-described examples. While values and numbers which are obtained by experiments and calculations are adequately set, the condition of a blood vessel may be determined. When the determination is performed while the number of thresholds is increased, the determination can be performed in a larger multistage and detailed manner.

In the example illustrated in FIG. 5, while 0.8 and 1.3 are set as the first and second thresholds, respectively, the determination is performed. With respect to the subjects P, Q, and S, the dABI is within the range of 0.8 to 1.3, and therefore it is determined that the condition of a blood vessel is normal. Consequently, an alarming display or the like is not particularly performed. On the other hand, with respect to the subject R, the dABI is 0.7, and equal to or smaller than the first threshold of 0.8, and therefore it is determined that there is a possibility of stenosis or occlusion of a blood vessel. Consequently, a broken line with squares is displayed in the periphery of the subject R. This display enables the user such as a medical staff to easily know that there is a possibility of stenosis or occlusion of the blood vessel of the subject R.

As described above, according to the index outputting apparatus 100 of the first embodiment, the dABI is calculated while the diastolic blood pressures of the upper and lower limbs of the living body are acquired, and the calculated dABI is output as an index for determining the condition of a blood vessel. The dABI is hardly affected by calcification of a blood vessel. Even when calcification occurs, therefore, it is possible to output an index from which the condition of a blood vessel can be adequately determined. When the user checks the dABI, for example, it is therefore possible to adequately determine the condition of a blood vessel even in the case where calcification occurs.

Moreover, the index outputting apparatus 100 determines the condition of the blood vessel of the living body based on the dABI. Therefore, the user can easily know the condition of the blood vessel. The condition of a blood vessel is automatically determined based on the calculated dABI, and therefore a necessary procedure in the next step can be automatically performed based on the determination result.

Moreover, the index outputting apparatus 100 determines the condition of a blood vessel by determining whether the dABI is equal to or smaller than the first threshold or equal to or larger than the second threshold. Therefore, it is possible to adequately determine whether there is an abnormality such as stenosis or occlusion in a blood vessel or not.

Moreover, the index outputting apparatus 100 displays the dABI. When the user checks the display, therefore, the user can easily know the condition of a blood vessel.

Moreover, the index outputting apparatus 100 employs the pressure raising method in which the cuff pressure is gradually raised from the state where the pressure is zero, as the method for measuring a blood pressure. In the embodiment, the diastolic blood pressure can be acquired at the timing when the mean blood pressure is acquired, and the condition of a blood vessel can be determined based on the dABI. Therefore, the cuff pressure is requested only to be raised to a value similar to the internal pressure of a blood vessel, and not required to be raised more than necessary. Also with respect to a subject in whom, for example, calcification occurs in the blood vessel and hence the blood vessel is easily broken, therefore, the condition of the blood vessel can be safely determined without imposing a load more than necessary to the blood vessel.

Moreover, the index outputting apparatus 100 outputs the dABI as an index for determining the condition of a blood vessel. It is found that, as compared with the systolic blood pressure, the diastolic blood pressure exhibits a value which is less dispersed depending on the measurement portion in the living body, and a stable result can be obtained without being affected by the measurement portion. When the dABI is used, therefore, an influence due to the measurement portion can be suppressed, and the condition of a blood vessel can be stably determined. Also with respect to a subject in whom, because of an injury or the like, it is difficult to measure the diastolic blood pressure in a specific portion such as the upper arm, the diastolic blood pressure can be measured in another portion such as the front arm, and the condition of a blood vessel can be adequately determined. Therefore, the determination can be flexibly performed in accordance with the condition of the subject.

Although, in the embodiment described above, the blood pressure acquiring section 170 communicates with the measuring devices such as cuffs, the invention is not limited to this. The blood pressure acquiring section 170 may include measuring devices such as cuffs.

Although, in the embodiment described above, the example in which the blood pressures of the upper arm and the ankle joint are acquired as those of the upper and lower limbs, respectively has been described, the invention is not limited to this. For example, the blood pressure of the front arm, the wrist, the hand finger, or the like may be acquired as that of the upper limb, and the blood pressure of the thigh, the lower leg, the foot finger, or the like may be acquired as that of the lower limb.

Although, in the embodiment described above, the blood pressure is measured by the pressure raising method in which the cuff pressure is gradually raised from zero, the invention is not limited to this. For example, the blood pressure may be measured by the pressure lowering method in which the cuff pressure is gradually lowered from a state where the cuff pressure is high, or the step pressure reducing method.

Although, in the embodiment described above, the diastolic blood pressure is measured from the cuff pressure at the timing when a pulse wave amplitude having a magnitude which is obtained by subtracting the predetermined rate from the maximum value of the pulse wave amplitude is detected, the invention is not limited to this. For example, the diastolic blood pressure may be measured by checking Korotkoff sounds by using a stethoscope or the like.

Although, in the embodiment described above, the calculated dABI is output by displaying it on the displaying section, the invention is not limited to this. For example, the dABI may be output in the form of sounds. Alternatively, data indicating the dABI may be transmitted to another analysis apparatus or the like. The dABI may be not only output as it is, but also output after converted to an index such as “High,” or “Low,” or to a message, sounds, or the like indicative of a determination result such as “Normal,” “Abnormal,” or “There is a possibility of stenosis or occlusion.”

### Example 2

Next, the index outputting apparatus 100 of a second embodiment will be described.

In the first embodiment described above, the index outputting apparatus 100 outputs the dABI as an index. However, the index output from the index outputting apparatus 100 is not limited to the dABI. For example, the ABI, the mABI, and the ppABI may be output in combination with the dABI. The second embodiment in which the ABI, the mABI, or the ppABI is output in combination with the dABI will be described.

The configuration of the index outputting apparatus 100 of the second embodiment is similar to that of the index outputting apparatus 100 of the first embodiment. Hereinafter, description of portions overlapping with those of the first embodiment is omitted, and description is made with emphasis on portions different from those of the first embodiment.

In the index outputting apparatus 100 of the second embodiment, the blood pressure acquiring section 170 acquires the systolic blood pressure, the mean blood pressure, and the pulse pressure in addition to the diastolic blood pressure.

The processes executed by the index outputting apparatus 100 of the second embodiment are basically identical with those executed by the index outputting apparatus 100 of the first embodiment and illustrated in the flowchart of FIG. 4. Hereinafter, processes which are different from those of the first embodiment will be described in detail.

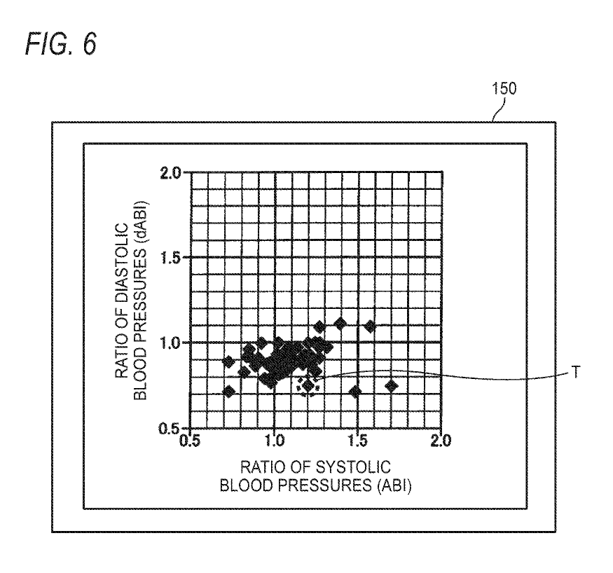

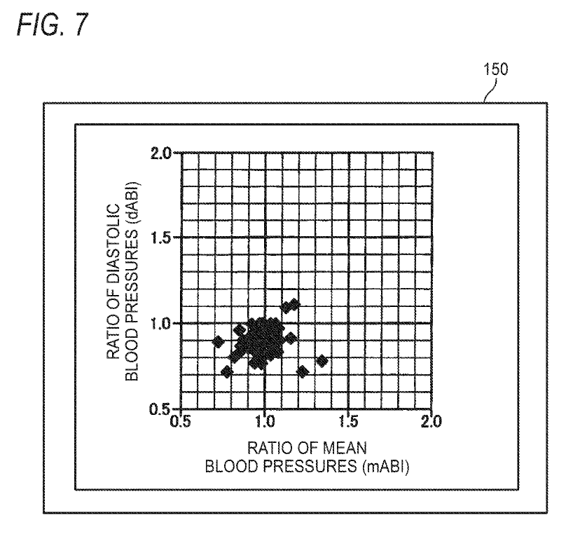

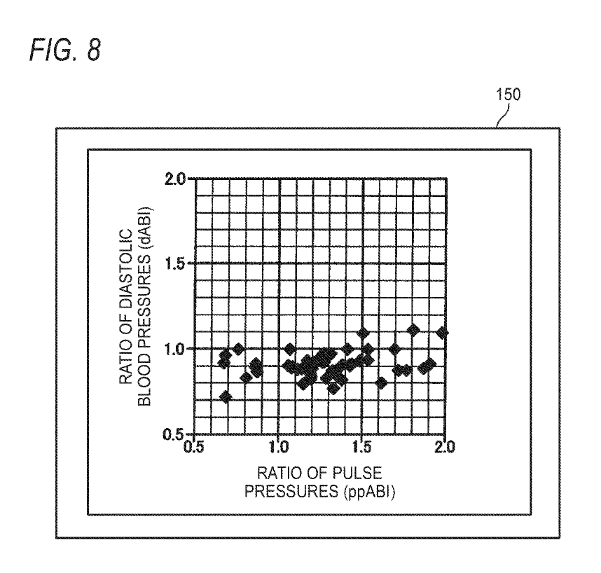

FIG. 6 illustrates an example of a screen which displays combinations of a ratio of diastolic blood pressures, and that of systolic blood pressures, FIG. 7 illustrates an example of a screen which displays combinations of a ratio of diastolic blood pressures, and that of mean blood pressures, and FIG. 8 illustrates an example of a screen which displays combinations of a ratio of diastolic blood pressures, and that of pulse pressures.

Firstly, the index outputting apparatus 100 acquires in the process of step S101 the diastolic blood pressure, systolic blood pressure, mean blood pressure, and pulse pressure of each of the upper and lower limbs of the subject. Methods of acquiring the diastolic blood pressures, the systolic blood pressures, the mean blood pressures, and the pulse pressures are identical with those of the above-described first embodiment.

Then, the index outputting apparatus 100 calculates in the process of step S102 the dABI, ABI, mABI, and ppABI which are ratios of the values of the lower limb to those of the upper limb.

Then, the index outputting apparatus 100 displays in the process of step S103 combinations of the dABI, and the ABI, the mABI, and the ppABI. Specifically, the index outputting apparatus 100 displays screens such as illustrated in FIGS. 6 to 8 on the displaying section 150. In the example of FIGS. 6 to 8, results of calculations for many subjects are plotted on a two-dimensional matrix. In the screen of FIG. 6, for example, the ordinate indicates a result of a calculation of a ratio of diastolic blood pressures, and the abscissa indicates a result of a calculation of a ratio of systolic blood pressures. The user such as a medical staff can determine the condition of a blood vessel of the subject from the position of a plot displayed on the screen. The determination method will be described in detail later. The index outputting apparatus 100 may switchingly display the screens of FIGS. 6 to 8 in accordance with instructions given by the user, or simultaneously display these screens.

Then, the index outputting apparatus 100 determines in the process of step S104 the condition of a blood vessel while combining the ABI, the mABI, and the ppABI with the dABI.

For example, the plot T which is enclosed by the broken line in the screen of FIG. 6 will be exemplarily described. With respect to the plot T. the ABI indicated by the abscissa is about 1.2. When the conventional determination method in which the ABI is used is applied, therefore, it is determined that the condition of a blood vessel is normal, because the ABI is within the range of 0.9 to 1.4. With respect to the plot T, however, the dABI indicated by the ordinate is about 0.75. When the determination method of the first embodiment in which the dABI is used is applied, therefore, it can be determined that there is a possibility of stenosis or occlusion, because the dABI is equal to or smaller than 0.8. Although the value of the dABI is small, moreover, the value of the ABI is large, and therefore it can be determined that there is a possibility of occurrence of calcification in the blood vessel of the lower limb. This is because, when calcification of a blood vessel occurs, the ABI is evaluated to be higher than the true value, and therefore the ABI is increased in the case where calcification occurs in the blood vessel of the lower limb. This determination method can be applied to the whole screen of FIG. 6 to determine the condition of a blood vessel based on the region where the plot is located. Hereinafter, the method for determining the condition of a blood vessel based on the plot region will be described in detail.

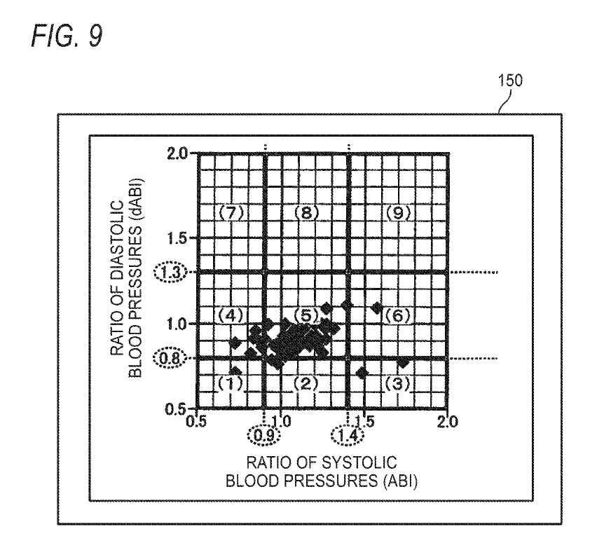

FIG. 9 illustrates an example of the method for determining the condition of a blood vessel based on the plot region.

As illustrated in FIG. 9, for example, the plot region is divided into nine regions of region (1) to region (9). The boundaries which divide the region in the longitudinal direction are set at positions of 0.9 and 1.4 as thresholds for determining the condition of a blood vessel by using the ABI. The boundaries which divide the region in the lateral direction are set at positions of 0.8 and 1.3 as thresholds for determining the condition of a blood vessel by using the dABI. Hereinafter, the determination method in the cases where plots are in the respective regions will be described.

In the case where the plot is in region (1), the ABI is equal to or smaller than 0.9. and the dABI is equal to or smaller than 0.8. In this case, in both the determination using the ABI and that using the dABI, it is determined that there is a possibility that stenosis or occlusion occurs in a blood vessel. Therefore, it can be determined that the possibility of stenosis or occlusion of a blood vessel is high.

In the case where the plot is in region (2), although the ABI is in the normal range of 0.9 to 1.4, the dABI is equal to or smaller than 0.8. In this case, because of a reason similar to that in the case of the above-described plot T of FIG. 6, it can be determined that there is a possibility that stenosis or occlusion occurs in a blood vessel, and there is a further possibility that calcification occurs.

In the case where the plot is in region (3), the ABI is equal to or larger than 1.4, and the dABI is equal to or smaller than 0.8. In this case, similarly with region (2), it can be determined that there is a possibility that stenosis or occlusion occurs in a blood vessel, and there is a further possibility that calcification occurs. Since the ABI is equal to or larger than 1.4 or namely larger than the case of region (2), it can be determined that the possibility that calcification occurs is higher.

In the case where the plot is in region (4), the ABI is equal to or smaller than 0.9, and the dABI is in the range of 0.8 to 1.3. In this case, in the determination using the ABI, it is determined that there is a possibility that stenosis or occlusion occurs in a blood vessel, and, in the determination using the dABI, it is determined that the condition is normal. Therefore, the results of the two determinations are different from each other, and hence it is determined that an abnormality of some kind occurs. A countermeasure such as a rerun of the examination or conduction of an examination of another kind may be taken.

In the case where the plot is in region (5), the ABI is in the normal range of 0.9 to 1.4, and the dABI is in the normal range of 0.8 to 1.3. In this case, in both the determination using the ABI and that using the diastolic blood pressure, it is determined that the condition of a blood vessel is normal. Therefore, it is possible to more surely determine that the condition of a blood vessel is normal.

In the case where the plot is in region (6), the ABI is equal to or larger than 1.4, and the dABI is in the normal range of 0.8 to 1.3. In this case, in the determination using the ABI, it is determined that there is a possibility that an abnormality such as calcification of a blood vessel occurs, and, in the determination using the dABI, it is determined that the condition is normal. Therefore, it can be determined that the possibility of stenosis or occlusion of a blood vessel is low, but there is a possibility that calcification of a blood vessel occurs.

In the case where the plot is in regions (7) to (9) the dABI is equal to or larger than 1.3, and therefore it is determined that an abnormality of some kind occurs in the condition of a blood vessel, the measurement method, the measuring device, etc. In this case, a countermeasure such as a rerun of the examination, checking of the measurement method and the measuring device, or an examination of another kind may be taken.

As described above, according to the index outputting apparatus 100 of the second embodiment, at least one of the ABI, the mABI, and the ppABI is displayed together with the dABI. Therefore, the user can simultaneously check different indexes, and determine the condition of a blood vessel more correctly and quickly.

Moreover, the index outputting apparatus 100 determines the condition of a blood vessel based on a combination of at least one of the ABI, the mABI, and the ppABI, and the dABI. Therefore, it is possible to determine the condition of a blood vessel in a combination of a plurality of determination methods using different indexes, and determination can be performed more correctly.

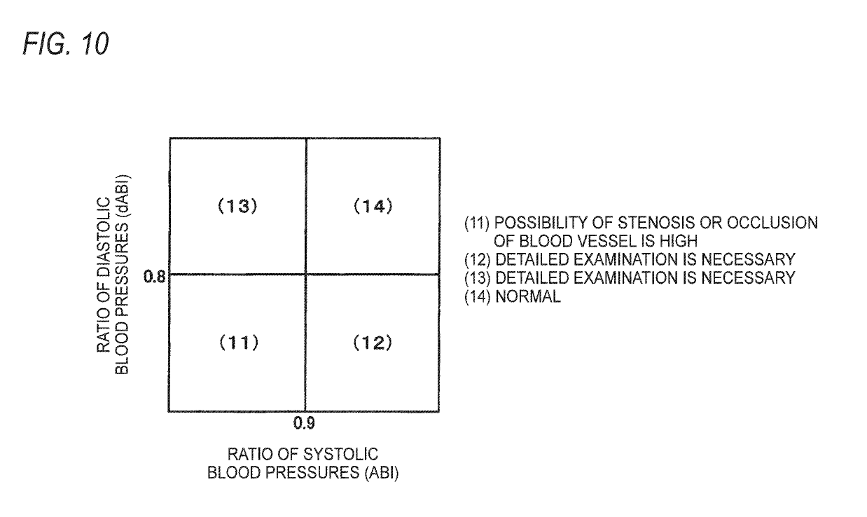

FIG. 10 illustrates an example of a method for determining the condition of a blood vessel based on a more convenient plot region which is assumed to be used in simple screening performed in a town clinic or the like, and which is limited to determination of stenosis or occlusion.

As illustrated in FIG. 10, the plot region is divided into, for example, four regions of region (11) to region (14). The boundary which divides the region in the longitudinal direction is set at a position of 0.9 as a threshold for determining the condition of a blood vessel by using the ABI. The boundary which divides the region in the lateral direction is set at a positions of 0.8 as a threshold for determining the condition of a blood vessel by using the dABI. Hereinafter, the determination method in the cases where plots are in the respective regions will be described.

In the case where the plot is in region (11), the ABI is equal to or smaller than 0.9, and the dABI is equal to or smaller than 0.8. In this case, in both the determination using the ABI and that using the dABI, it is determined that there is a possibility that stenosis or occlusion occurs in a blood vessel. Therefore, it can be determined that the possibility of stenosis or occlusion of a blood vessel is high.

In the case where the plot is in region (12), the ABI is equal to or larger than 0.9, and the dABI is equal to or smaller than 0.8. In this case, it is determined that there is a possibility that stenosis or occlusion occurs in a blood vessel, and a detailed examination is necessary.

In the case where the plot is in region (13), the ABI is equal to or smaller than 0.9, and the dABI is equal to or larger than 0.8. In this case, in the determination using the ABI, it is determined that there is a possibility that stenosis or occlusion occurs in a blood vessel, and a detailed examination is necessary.

In the case where the plot is in region (14), the ABI is equal to or larger than 0.9, and the dABI is equal to or larger than 0.8.

Although, in the embodiment described above, the determination is performed while the plot region is divided into the nine or four regions, the invention is not limited to this. The number and positions of the divided regions may be adequately changed in accordance with the number and values of thresholds which are set in order to determination for each index.

Although, in the embodiment described above, results of calculations of the two indexes are plotted on a two-dimensional matrix, the invention is not limited to this. For example, results of calculations of three indexes may be plotted on a three-dimensional matrix, or four indexes may be displayed in the form of a bar graph or a rectangular graph.

The means and methods for performing the various processes in the index outputting apparatuses of the above-described embodiments may be realized by either of a dedicated hardware circuit and a programmed computer. For example, the programs may be provided by a computer readable recording medium such as a flexible disk or a CD-ROM, or provided online through a network such as the Internet. In this case, the programs which are recorded on a computer readable recording medium are usually transferred and stored in a storage section such as a hard disk drive. Alternatively, the programs may be provided in the form of standalone application software, or incorporated as one function of the index outputting apparatus into software of the apparatus.

The present application is based on Japanese Patent Application No. 2015-099352, filed on May 14, 2015, the entire contents of which are incorporated herein by reference.

### INDUSTRIAL APPLICABILITY

There is provided with a method, apparatus, and program for outputting an index for adequately determining the condition of a blood vessel even in the case where calcification of the blood vessel occurs.

### REFERENCE SIGNS LIST

100 index outputting apparatus

110 CPU

120 ROM

130 RAM

140 storage device

150 displaying section

160 operating section

170 blood pressure acquiring section

180 bus

What is claimed is:

1-10. (canceled)

11. A method for outputting an index for determining a condition of a blood vessel comprising:
acquiring a diastolic blood pressure for each of upper and lower limbs of a living body;
calculating a ratio of the diastolic blood pressure acquired in the acquiring step of the lower limb to the diastolic blood pressure acquired in the acquiring step of the upper limb; and
outputting the ratio of the diastolic blood pressures calculated in the calculating step,
wherein, in the outputting step, the ratio of the diastolic blood pressures which is calculated in the calculating step is displayed,
in the acquiring step, at least one of a systolic blood pressure, a mean blood pressure, and a pulse pressure is further acquired for each of the upper and lower limbs of the living body,
in the calculating step, at least one of a ratio of the systolic blood pressure of the lower limb to the systolic blood pressure of the upper limb, a ratio of the mean blood pressure of the lower limb to the mean blood pressure of the upper limb, and a ratio of the pulse pressure of the lower limb to the pulse pressure of the upper limb is further calculated, and,
in the outputting step, at least one of the ratio of the systolic blood pressures, the ratio of the mean blood pressures, and the ratio of the pulse pressures is displayed together with the ratio of the diastolic blood pressures, the ratio of the systolic blood pressures, the ratio of the mean blood pressures, and the ratio of the pulse pressures being calculated in the calculating step.

12. The method according to claim 11 further comprising:
determining a condition of a blood vessel based on the ratio of the diastolic blood pressures calculated in the calculating step.

13. The method for outputting an index according to claim 12, wherein, in the determining step, the condition of a blood vessel is determined by determining whether the ratio of the diastolic blood pressures which is calculated in the calculating step is equal to or smaller than a first threshold, or equal to or larger a second threshold that is larger than the first threshold.

14. The method for outputting an index according to claim 12, wherein, in the determining step, the condition of a blood vessel is determined based on a combination of at least one of the ratio of the systolic blood pressures, the ratio of the mean blood pressures, and the ratio of the pulse pressures, and the ratio of the diastolic blood pressures.

15. An apparatus for outputting an index for determining a condition of a blood vessel comprising:
an acquiring section that acquires a diastolic blood pressure for each of upper and lower limbs of a living body;
a calculating section that calculates a ratio of the diastolic blood pressure of the lower limb to the diastolic blood pressure of the upper limb, the diastolic blood pressure of the lower limb and the diastolic blood pressure of the upper limb being acquired by the acquiring section; and
an outputting section that outputs the ratio of the diastolic blood pressures that is calculated by the calculating section,
wherein the outputting section displays the ratio of the diastolic blood pressures which is calculated by the calculating section,
the acquiring section further acquires at least one of a systolic blood pressure, a mean blood pressure, and a pulse pressure for each of the upper and lower limbs of the living body,
the calculating section further calculates at least one of a ratio of the systolic blood pressure of the lower limb to the systolic blood pressure of the upper limb, a ratio of the mean blood pressure of the lower limb to the mean blood pressure of the upper limb, and a ratio of the pulse pressure of the lower limb to the pulse pressure of the upper limb, and,
the outputting section displays at least one of the ratio of the systolic blood pressures, the ratio of the mean blood pressures, and the ratio of the pulse pressures together with the ratio of the diastolic blood pressures, the ratio of the systolic blood pressures, the ratio of the mean blood pressures, and the ratio of the pulse pressures being calculated by the calculating section.

16. A non-transitory computer readable medium storing a program causing a computer to execute a process for outputting an index for determining a condition of a blood vessel, the process comprising:
acquiring a diastolic blood pressure for each of upper and lower limbs of a living body;
calculating a ratio of the diastolic blood pressure of the lower limb to the diastolic blood pressure of the upper limb, the diastolic blood pressure of the lower limb and the diastolic blood pressure of the upper limb being acquired in the acquiring step; and
outputting the ratio of the diastolic blood pressures which is calculated in the calculating step,
wherein, in the outputting step, the ratio of the diastolic blood pressures which is calculated in the calculating step is displayed,
in the acquiring step, at least one of a systolic blood pressure, a mean blood pressure, and a pulse pressure is further acquired for each of the upper and lower limbs of the living body,
in the calculating step, at least one of a ratio of the systolic blood pressure of the lower limb to the systolic blood pressure of the upper limb, a ratio of the mean blood pressure of the lower limb to the mean blood pressure of the upper limb, and a ratio of the pulse pressure of the lower limb to the pulse pressure of the upper limb is further calculated, and,
in the outputting step, at least one of the ratio of the systolic blood pressures, the ratio of the mean blood pressures, and the ratio of the pulse pressures is displayed together with the ratio of the diastolic blood pressures, the ratio of the systolic blood pressures, the ratio of the mean blood pressures, and the ratio of the pulse pressures being calculated in the calculating step.
